(19) United States
(12) Patent Application Publication
(10) Pub. No.: US 2019/0350556 A1
(43) Pub. Date: Nov. 21, 2019

(54) ULTRASOUND DIAGNOSTIC APPARATUS AND NON-TRANSITORY COMPUTER-READABLE RECORDING MEDIUM STORING CONTROL PROGRAM THEREIN

(71) Applicant: Konica Minolta, Inc., Tokyo (JP)

(72) Inventor: Shinya KUROKAWA, Tokyo (JP)

(21) Appl. No.: 16/409,202

(22) Filed: May 10, 2019

(30) Foreign Application Priority Data
May 21, 2018 (JP) 2018-097362

(51) Int. Cl. A61B 8/00; A61B 8/08

(52) U.S. Cl. CPC A61B 8/468; A61B 8/465; A61B 8/463; A61B 8/0866

(57) ABSTRACT

Provided are an ultrasound diagnostic apparatus capable of reducing examination time when a plurality of preborn children exist in a body of the same mother. The ultrasound diagnostic apparatus includes: a measurer that measures a predetermined item for each of the plurality of preborn children in the body of the same mother; a display; and a hardware processor that causes the display to display measurement results respectively allocated to the plurality of preborn children, the hardware processor causing an operation key associated with an operation for changing the allocation of the measurement results to be displayed on a same screen as a screen on which the measurement results are displayed, the hardware processor changing the allocation of the measurement results respectively allocated to the plurality of preborn children among the plurality of preborn children in response to an operation of the operation key.

### CROSS REFERENCE TO RELATED APPLICATIONS

The entire disclosure of Japanese patent Application No. 2018-097362 filed on May 21, 2018, is incorporated herein by reference in its entirety.

### BACKGROUND

### Technological Field

The present invention relates to an ultrasound diagnostic apparatus and a non-transitory computer-readable recording medium storing a control program therein.

### Description of Related Art

An ultrasound diagnostic apparatus includes, for example, an ultrasound probe that transmits an ultrasound pulse into a subject and receives an ultrasound echo from the inside of the subject, an ultrasound diagnostic apparatus main body that generates an ultrasound image on the basis of the received ultrasound echo, and a monitor that displays the generated ultrasound image.

For example, Japanese Patent No. 4701011 (Patent Literature 1) discloses an ultrasound diagnostic apparatus including measured value acquisition means for acquiring a measured value of a preborn child on the basis of an ultrasound image displayed on a monitor.

For example, in the measurement of twin preborn children, the ultrasound probe is placed on the body of the mother to receive an ultrasound echo from the inside of the body of the mother, and an ultrasound image is generated on the basis of the received ultrasound echo. Then, the generated ultrasound image is displayed on the monitor. The measured value acquisition means performs measurement of items (for example, the head biparietal diameter (BPD) and the like of the preborn children) that are indicators for the development of the preborn children on the basis of the ultrasound image.

The preborn child is given identification information (for example, “preborn child A” or “preborn child B”) for distinguishing the preborn child from the other preborn child in the body of the same mother. The doctor selects a preborn child before the measurement by specifying and inputting identification information (“preborn child A” or “preborn child B”). That is, after one of the preborn children A and B is selected, the measurement of the selected preborn child is performed.

The preborn child measurement is performed a plurality of times in an almost periodic manner, and a measurement result is obtained every time the preborn child measurement is performed. The measurement result is recorded in a recording area for each of preborn child A and preborn child B of a patient (the body of the mother) in an obstetrics patient data base. The measurement result of the past is also recorded in the recording area. The report is used in a growth curve display function and the like included in the ultrasound diagnostic apparatus. The growth curve display function displays the average growth curve and the growth curve of the measurement target in comparison with each other, or displays a mark indicating the current measured value (the measured value of the present measurement) of the preborn child on the growth curve, for example.

For example, in Japanese Patent No. 4322370 (Patent Literature 2), an ultrasound diagnostic apparatus that automatically allocates each measurement result to one of the plurality of preborn children on the basis of a predetermined condition is disclosed.

Meanwhile, in order to generate a report describing the development of the plurality of preborn children, the plurality of preborn children need to be accurately distinguished in every measurement. It takes time in order to accurately distinguish the preborn children. If the measurement is performed while the preborn children are erroneously distinguished, the measurement of the preborn children needs to be redone from the start. Naturally, the redoing of the preborn child measurement extends the examination time.

In the ultrasound diagnostic apparatus disclosed in Patent Literature 1 above, the measurement is started after preborn child A and preborn child B are selected. Therefore, when the positions of preborn child A and preborn child B in the body of the mother change due to movement and the like after the selection, the measured value of preborn child A may be associated with preborn child B, and the measured value of preborn child B may be associated with preborn child A. In this case, the measurement of the preborn children needs to be redone from the start, and the examination time is extended.

In the ultrasound diagnostic apparatus disclosed in Patent Literature 2 above, the measurement results are automatically allocated to the preborn children. Therefore, when the measurement results are approximate to each other, the measurement results may be erroneously allocated to the preborn children.

### SUMMARY

A first object of the present invention is to provide an ultrasound diagnostic apparatus and a control program capable of reducing examination time when a plurality of preborn children exist in a body of the same mother. A second object of the present invention is to provide an ultrasound diagnostic apparatus and a control program capable of suppressing erroneous association between preborn children and measurement results when a plurality of preborn children exist in a body of the same mother.

To achieve at least one of the abovementioned objects, according to an aspect of the present invention, an ultrasound diagnostic apparatus reflecting one aspect of the present invention comprises: a measurer that measures a predetermined item for each of a plurality of preborn children in a body of a same mother; a display; and a hardware processor that causes the display to display measurement results respectively allocated to the plurality of preborn children, the hardware processor causing an operation key associated with an operation for changing the allocation of the measurement results to be displayed on a same screen as a screen on which the measurement results are displayed, the hardware processor changing the allocation of the measurement results respectively allocated to the plurality of preborn children among the plurality of preborn children in response to an operation of the operation key.

To achieve at least one of the abovementioned objects, according to an aspect of the present invention, a non-transitory computer-readable recording medium storing therein a control program reflecting one aspect of the present invention, the program causing a computer of an ultrasound diagnostic apparatus comprising a measurer that measures a predetermined item for each of a plurality of preborn children in a body of a same mother and a display to execute: causing the display to display measurement results respectively allocated to the plurality of preborn children; and causing an operation key associated with an operation for changing the allocation of the measurement results to be displayed on a same screen as a screen on which the measurement results are displayed, and changing the allocation of the measurement results respectively allocated to the plurality of preborn children among the plurality of preborn children in response to an operation of the operation key.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a block diagram illustrating the configuration of an ultrasound diagnostic apparatus according to Embodiment 1 of the present invention;

FIG. 2 illustrates an example of a display screen of a display section of the ultrasound diagnostic apparatus;

FIG. 3 illustrates an example of a graph screen of the display section of the ultrasound diagnostic apparatus;

FIG. 4 is a flowchart illustrating an example of processing of changing the allocation of measured values of preborn children;

FIG. 5 illustrates another example of an operation key;

FIG. 6 illustrates an example of likelihood information:

FIG. 7 is a block diagram illustrating the configuration of a multiple pregnancy development measurement function unit according to Embodiment 2 of the present invention:

FIG. 8 illustrates an example of a message:

FIG. 9 illustrates another example of a message;

FIG. 10 is a flowchart illustrating an example of processing of changing the allocation of the measured values of the preborn children;

FIG. 11 is a block diagram illustrating the configuration of a multiple pregnancy development measurement function unit according to Embodiment 3 of the present invention; and

FIG. 12 illustrates an example of allocation change information.

### DETAILED DESCRIPTION OF EMBODIMENTS

Hereinafter, one or more embodiments of the present invention will be described with reference to the drawings. However, the scope of the invention is not limited to the disclosed embodiments.

### Embodiment 1

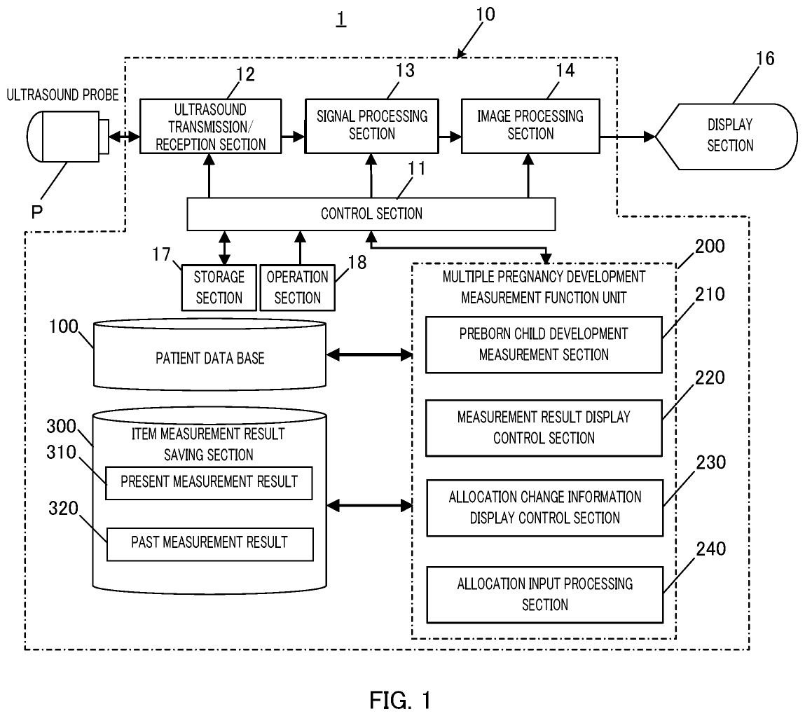

Embodiment 1 of the present invention is described below with reference to the accompanying drawings. FIG. 1 is a block diagram illustrating the configuration of ultrasound diagnostic apparatus 1 according to Embodiment 1 of the present invention.

As illustrated in FIG. 1, ultrasound diagnostic apparatus 1 includes ultrasound diagnostic apparatus main body 10 and ultrasound probe P.

Ultrasound probe P transmits ultrasound to a subject such as a living body (not shown), and receives a reflected wave (reflected echo) of the ultrasound reflected on the subject. Then, ultrasound probe P acquires a reception signal that is an electrical signal from the received reflected echo, and outputs the reception signal to ultrasound diagnostic apparatus main body 10.

Ultrasound diagnostic apparatus main body 10 is connected to ultrasound probe P via a cable and the like. Ultrasound diagnostic apparatus main body 10 transmits a transmission signal of an electrical signal to ultrasound probe P and causes ultrasound probe P to transmit ultrasound to the subject. Ultrasound diagnostic apparatus main body 10 images and displays the internal state in the subject as an ultrasound image on the basis of the reception signal received from ultrasound probe P.

As illustrated in FIG. 1, ultrasound diagnostic apparatus main body 10 includes control section 11, ultrasound transmission/reception section 12, signal processing section 13, image processing section 14, a frame memory (not shown), storage section 17, operation section 18, and the like.

Control section 11 includes a Central Processing Unit (CPU), a Random Access Memory (RAM), and the like, executes various types of processing in cooperation with various types of programs stored in storage section 17, and comprehensively controls the operation of ultrasound diagnostic apparatus 1.

Ultrasound transmission/reception section 12 supplies a transmission signal to ultrasound probe P for causing ultrasound probe P to generate ultrasound. Ultrasound transmission/reception section 12 receives a reflected echo generated in the subject by ultrasound probe P, generates a reception signal, and outputs the reception signal to signal processing section 13.

Signal processing section 13 electrically amplifies a reception electrical signal, which is a converted version of the reception signal, and outputs the reception electrical signal to image processing section 14 after applying signal processing such as digital conversion.

Image processing section 14 generates a B-mode image on the basis of the digital reception signal output from signal processing section 13. The B-mode image expresses the intensity of the reception signal by brightness. The B-mode image generated as above is output to a memory section (not shown) as an ultrasound image. The memory section includes a frame memory (not shown) that stores therein the ultrasound image output from image processing section 14 in units of frame images. Once the ultrasound image is output from image processing section 14, control section 11 overwrites the output ultrasound image on the frame memory in units of frames, and causes the ultrasound image to be displayed on display section 16.

Display section 16 is made up of a Liquid Crystal Display (LCD), a Cathode-Ray Tube (CRT) display, an organic Electronic Luminescence (EL) display, a plasma display, or the like. Display section 16 sequentially displays the ultrasound images stored in the frame memory (not shown) in a continuous manner in accordance with the control from control section 11. Display section 16 displays various types of operation screens and the like on the screen in accordance with the control from control section 11.

Storage section 17 is made up of a Hard Disk Drive (HDD), a semiconductor nonvolatile memory, and the like, for example. Storage section 17 stores therein various types of program and data necessary for the execution of the programs, and the like.

Operation section 18 includes various types of switches, function buttons, a trackball, a mouse, a keyboard, a touch panel, and the like. Operation section 18 outputs operation signals to control section 11.

Ultrasound diagnostic apparatus 1 includes patient data base 100, multiple pregnancy development measurement function unit 200, and item measurement result saving section 300 (corresponding to a “measurement result recording section” of the present invention).

Patient data base 100 records patient information. As the patient information, there are items such as a “UID”, “shooting time and date”, an “examination ID”, a “shooting part”, a “patient ID”, a “patient name”, an “age”, a “gender”, and an “image saving destination”. Patient data base 100 records the ultrasound image in association with the patient information.

Multiple pregnancy development measurement function unit 200 includes preborn child development measurement section 210 (corresponding to a “measurement section” in the present invention), measurement result display control section 220, and allocation changing section (described below) and allocation change information display control section 230. Multiple pregnancy development measurement function unit 200 performs measurement of the items serving as indicators for the development of the plurality of preborn children in the body of the same mother with use of the ultrasound image.

Preborn child development measurement section 210 measures items such as the head biparietal diameter (BPD), the crown-rump length (CRL), the femur length (FL), the child body weight of the preborn child. The BPD is described below as a representative of the measured items of the preborn child. Note that the measurement result of the BPD may be simply referred to as a “measurement result”. The measured value of the BPD may be simply referred to as a “measured value”.

An example of a measurement method performed by preborn child development measurement section 210 is described. For example, the ultrasound image is frozen by operation section 18 in a state in which an image of a part that is desired to be measured is displayed on display section 16. As means for performing the freezing, an example in which operation section 18 is used is described, but the freezing may be performed by audio controls or automatically. A cursor for measurement is caused to be displayed on display section 16 by operation section 18. While the part that is desired to be measured is displayed on the frozen image, operating operation section 18 is operated to move the cursor for measurement is moved, specify the starting point and the ending point of the measurement, and designate a measurement range.

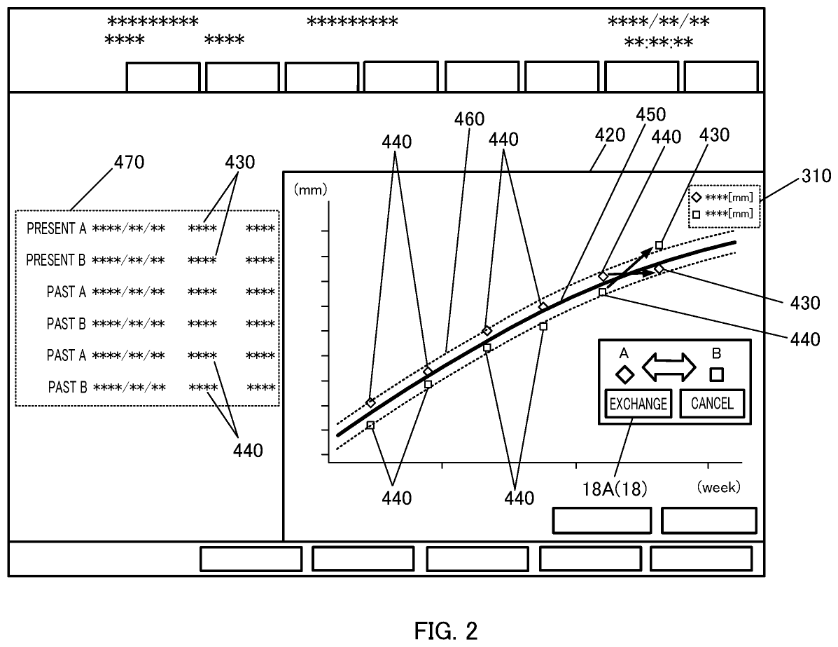

FIG. 2 illustrates an example of a display screen of display section 16. As illustrated in FIG. 2, measurement result display control section 220 causes display section 16 to display graph screen 420 in order to cause present measured values 430 to be displayed in a graph. On graph screen 420, a graph, in which present measured values 430 and past measured values 440 when there are measured values measured in the past are plotted, is displayed with the horizontal axis being the weeks of pregnancy [week] and the vertical axis being the length [mm]. On graph screen 420, average development curve 450 and statistical deviation curve 460 (for example, a +1.55 SD curve and a −1.55 SD curve) indicating the average development degree of the preborn child are displayed.

As illustrated in FIG. 2, measurement result display control section 220 causes display section 16 to display present measured values 430 and past measured values 440 as measured value display screen 470.

Item measurement result saving section 300 records present measured values 430 as present measurement result 310 together with the identification information for identifying the preborn children, the examination date and time, and the weeks of pregnancy of the preborn children.

Item measurement result saving section 300 also records previous or before (past) measured values 440 as past measurement result 320 together with the identification information, the examination date and time, and the weeks of pregnancy of the preborn children.

In the measurement of the plurality of preborn children, present measured values 430 may be approximate to each other. In this case, there is a fear that the measured values 430 are erroneously allocated to the preborn children.

In this embodiment, the allocation changing section that changes the allocation of plurality of measured values 430 among the plurality of preborn children is included. As illustrated in FIG. 1 and FIG. 2, the allocation changing section includes operation key 18A and allocation input processing section 240, for example.

As illustrated in FIG. 2, operation key 18A is displayed on graph screen 420. Allocation input processing section 240 changes the allocation of present measured values 430 (exchanges measured values 430) between preborn child A and preborn child B on the basis of a click operation of operation key 18A. Multiple pregnancy development measurement function unit 200 causes item measurement result saving section 300 to record changed present measured values 430 as present measurement result 310, and causes allocation change information indicating that the allocation is changed to be recorded in association with present measured values 430. Measurement result display control section 220 causes changed present measured values 430 to be displayed on graph screen 420.

Meanwhile, when the allocation of measured values 430 is changed, the allocation change information indicating that the allocation of measured values 430 has been changed is preferred to be left so that a user pays attention to the fact that paid measured values 430 are changed.

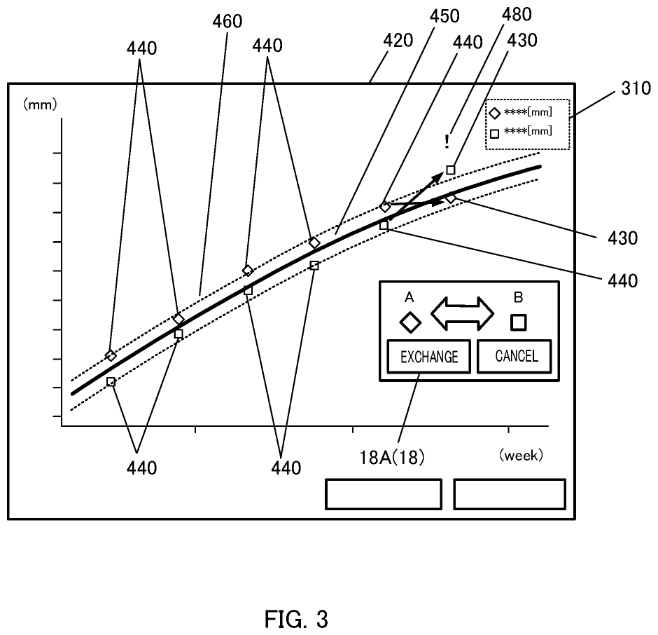

In this embodiment, allocation change information display control section 230 that causes the allocation change information to be displayed when the allocation of measured values 430 is changed is included. As illustrated in FIG. 3, allocation change information display control section 230 causes exclamation mark 480B serving as the allocation change information to be displayed near the display positions of measured values 430 the allocation of which is changed (near the plots indicating measured values 430) in graph screen 420. Note that allocation change information display control section 230 may cause exclamation mark 480B to be displayed near the display positions of measured values 430 in measured value display screen 470. As a result, the user can be prompted to visually recognize that the allocation of measured values 430 displayed near exclamation mark 480B has been changed.

When the allocation of the past measured values is changed, the allocation change information is preferred to be displayed also near display positions of the past measured values.

A configuration in which the allocation change for the preborn children can be performed not only for present measured values 430 but also for past measured values 440 is preferred. When the allocation change for the preborn children is performed for past measured values 440, past measured values 440 the allocation of which is desired to be changed are selected by the touch panel, the trackball, and the like (not shown), and the allocation for the preborn children is changed by operating operation key 18A.

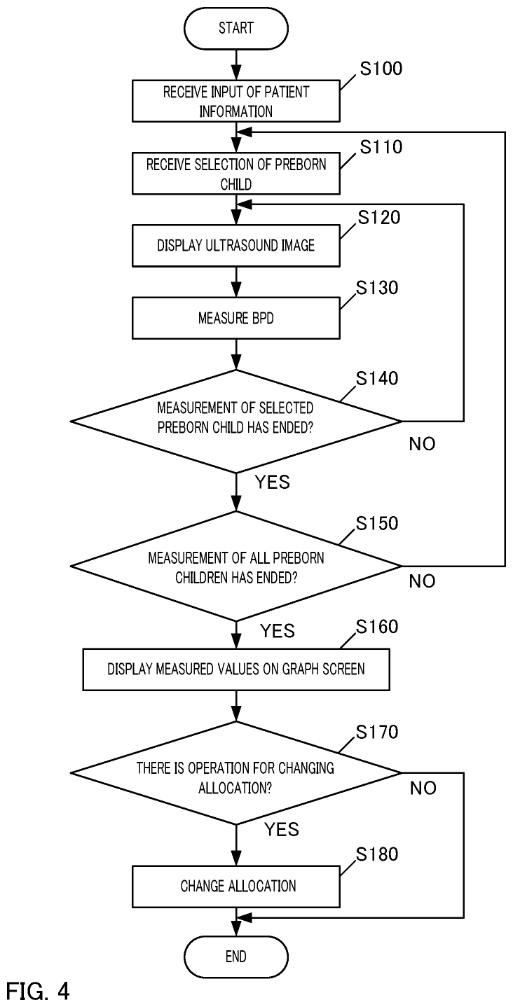

Next, processing from the input of the patient information to the change of the allocation of measured values 430 of the preborn children is described with reference to FIG. 4. FIG. 4 is a flowchart illustrating an example of the processing from the input of the patient information to the change of the allocation of measured values 430 of the preborn children.

In Step S100, the input of the patient information is received by operation section 18.

In Step S110, the selection of the preborn child is received by operation section 18.

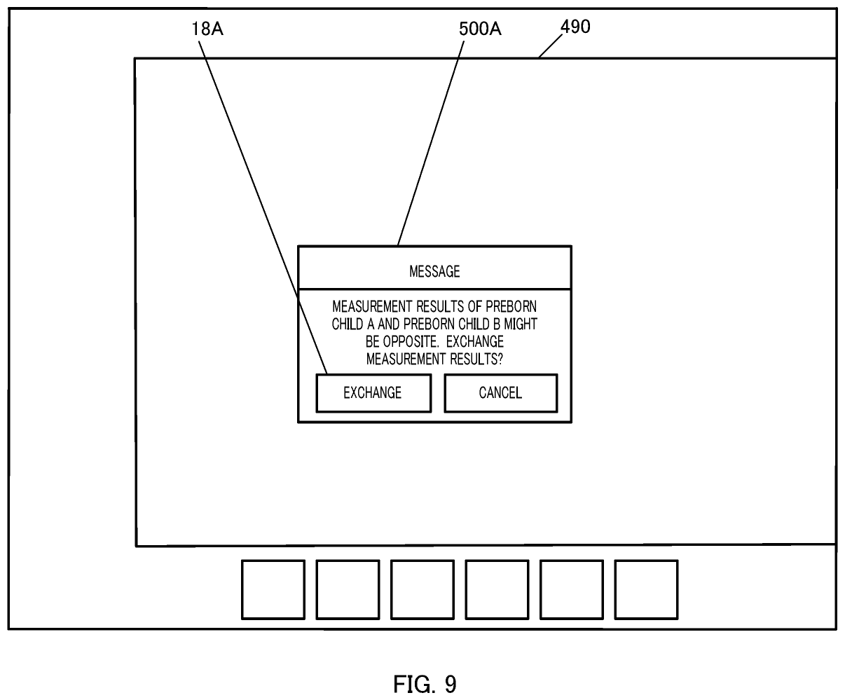

In Step S120, ultrasound image 490 (see FIG. 9) is displayed on display section 16 by operation section 18.

In Step S130, preborn child development measurement section 210 performs measurement of the BPD of the selected preborn child with use of ultrasound image 490.

In Step S140, preborn child development measurement section 210 determines whether the measurement of the selected preborn child has ended. When the measurement of the preborn child has ended (Step S140: YES), the processing transitions to Step S150. When the measurement of the preborn child has not ended (Step S140: NO), the processing transitions to Step S120.

In Step S150, preborn child development measurement section 210 determines whether the measurement of all the preborn children has ended. When the measurement of all the preborn children has ended (Step S150: YES), the processing transitions to Step S160. When the measurement of all the preborn children has not ended (Step S150: NO), the processing returns to Step S110.

In Step S160, measurement result display control section 220 causes measured values 430 to be displayed on graph screen 420.

In Step S170, it is determined whether there is an operation for changing the allocation of measured values 430. When there is an operation for changing the allocation (Step S170: YES), the processing transitions to Step S180. Meanwhile, when there is no operation for changing the allocation (Step S170: NO), the processing ends.

In Step S180, allocation input processing section 240 changes the allocation of measured values 430.

According to ultrasound diagnostic apparatus 1 according to the embodiment above, preborn child development measurement section 210 that measures a predetermined item for each of the plurality of preborn children in the body of the same mother, measurement result display control section 220 that causes measured values 430 of each of the plurality of preborn children to be displayed, and allocation input processing section 240 that changes the allocation of measured values 430 among the plurality of preborn children on the basis of the click operation of operation key 18A are included. As a result, the doctor can change the allocation of measured values 430 between preborn child A and preborn child B by only clicking on operation key 18A. As a result, the allocation of measured values 430 can be easily changed. Therefore, the time spent in the change of the allocation of measured values 430 decreases, and the examination time can be reduced.

According to the embodiment above, operation key 18A is displayed on graph screen 420. Therefore, the doctor can change the allocation of measured values 430 on the same screen as graph screen 420. As a result, the allocation of measured values 430 can be changed in an easier manner. Therefore, the time spent in the change of the allocation of measured values 430 decreases even more, and the examination time can be further reduced.

According to the embodiment above, when the allocation of measured values 430 is changed by the click operation of operation key 18A, allocation change information display control section 230 causes exclamation mark 480B serving as the allocation change information to be displayed near measured values 430. As a result, it is expected that the attention of the user is continuously payed to the fact that measured values 430 are changed.

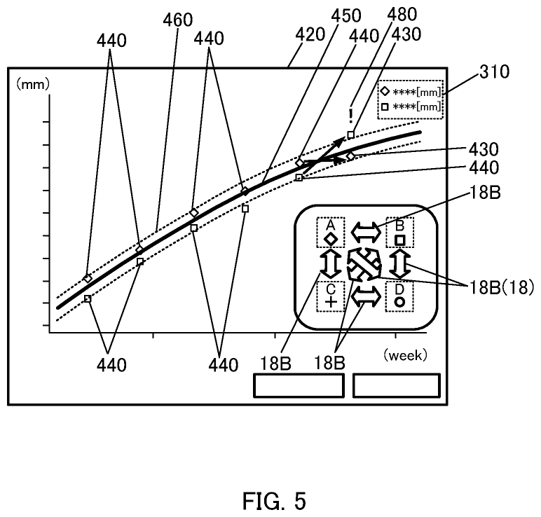

In the embodiment above, the allocation changing section includes operation key 18A for changing the allocation of measured values 430 between preborn child A and preborn child B. However, the present invention is not limited thereto. The allocation changing section may include an operation key for changing the allocation of measured values 430 among three or more preborn children. For example, as operation keys 18B illustrated in FIG. 5, the allocation of measured values 430 can be changed between preborn child A, preborn child B, preborn child C, and preborn child D.

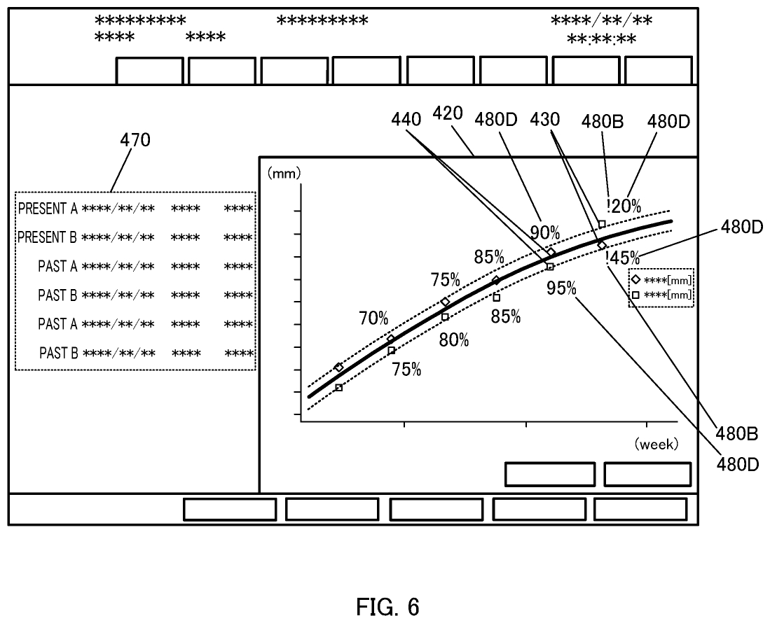

In the embodiment above, measurement result display control section 220 may cause likelihood information 480D indicating the likelihood of present measured value 430 belonging to the allocated preborn child to be displayed on graph screen 420 (see FIG. 6). For example, preborn child development measurement section 210 calculates likelihood information 480D indicating the degree by which present measured value 430 is deviated from an approximate curve (graph) based on past measured value 440 (rate of divergence). Likelihood information 480D is expressed in a percentage. The rate of divergence increases herein as the percentage decreases.

Measurement result display control section 220 causes likelihood information 480D to be displayed in correspondence to the plot indicating present measured value 430. By displaying likelihood information 480D, likelihood information 480D serves as a beneficial basis for decision for the user to decide whether the allocation of measured values 430 needs to be changed, and a case where the preborn children and the measurement results are erroneously associated with each other can be suppressed.

Multiple pregnancy development measurement function unit 200 may cause item measurement result saving section 300 to record likelihood information 480D in association with present measured value 430. When the next measured value is to be displayed, measurement result display control section 220 causes likelihood information 480D recorded in item measurement result saving section 300 to be displayed as past likelihood information. As a result, the tendency of the likelihood information can be presented to the user.

### Embodiment 2

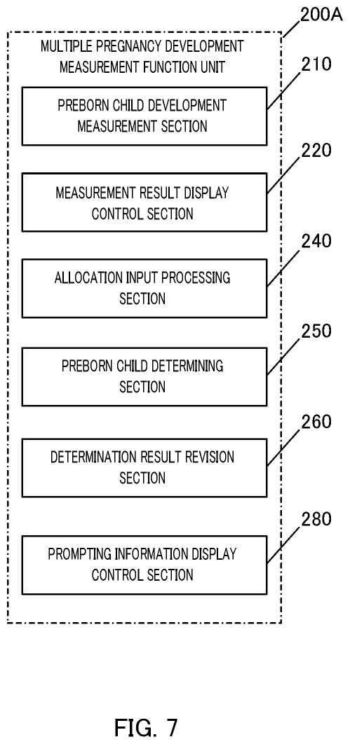

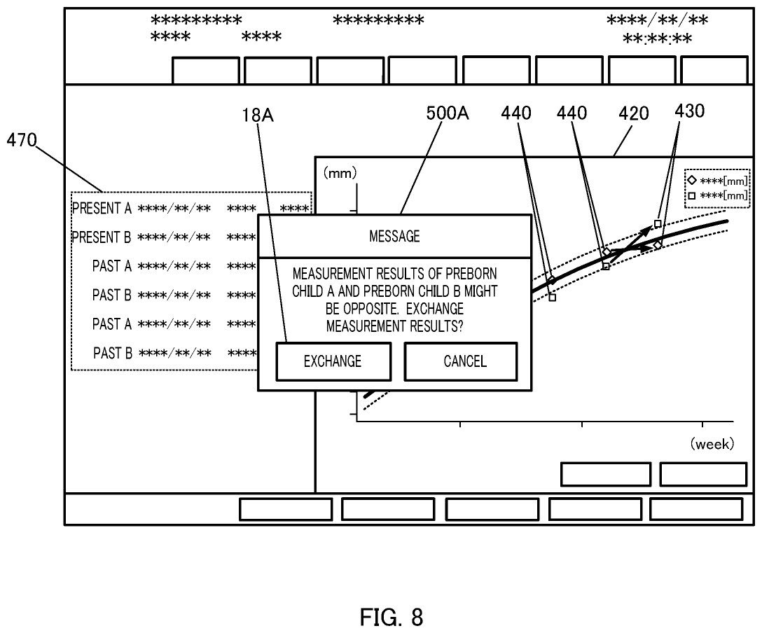

Next, Embodiment 2 of the present invention is described with reference to FIG. 7 and FIG. 8. FIG. 7 is a block diagram illustrating the configuration of multiple pregnancy development measurement function unit 200A of ultrasound diagnostic apparatus 1 according to Embodiment 2 of the present invention. In the description of Embodiment 2, the configurations different from those of Embodiment 1 are mainly described. The same configurations as those of Embodiment 1 are denoted by the same numerals and the description thereof is omitted.

As illustrated in FIG. 7, multiple pregnancy development measurement function unit 200A includes preborn child determining section 250 that determines whether the measurement results of the plurality of preborn children measured by preborn child development measurement section 210 belong to the respective preborn children. Preborn child determining section 250 determines whether measured values 430 (see FIG. 8) belong to the preborn children on the basis of predetermined conditions. The determination result of preborn child determining section 250 is recorded in item measurement result saving section 300.

A first predetermined condition is a slope of the past measured values. For example, preborn child determining section 250 determines that present measured value 430 of preborn child A does not belong to preborn child A when a difference between a slope of the past measured values (a linear slope connecting measured value 440 before last and previous measured value 440) and a slope of present measured values (a linear slope connecting previous measured value 440 of preborn child A and present measured value 430 of preborn child A) exceeds a predetermined permissible range.

A second predetermined condition is a case in which present measured value 430 is smaller than past measured value 440. For example, preborn child determining section 250 determines that present measured value 430 of preborn child A does not belong to preborn child A when present measured value 430 of preborn child A is smaller than past measured value 440 of preborn child A.

A third predetermined condition is the magnitude relationship between measured value 430 of preborn child A and measured value 430 of preborn child B. For example, when past measured value 440 of preborn child A is larger than past measured value 440 of preborn child B, preborn child determining section 250 determines that present measured value 430 of preborn child A and present measured value 430 of preborn child B do not belong to the respective preborn children (there is a possibility that the present measured values are opposite) when present measured value 430 of preborn child A is smaller than present measured value 430 of preborn child B.

As illustrated in FIG. 8, when at least one of the first to third conditions is satisfied, prompting information display control section 280 causes prompting information 500A that prompts the change of the allocation of present measured values 430 between preborn child A and preborn child B to be displayed on graph screen 420. Note that, as illustrated in FIG. 9, prompting information display control section 280 may display message 500A described above on ultrasound image 490.

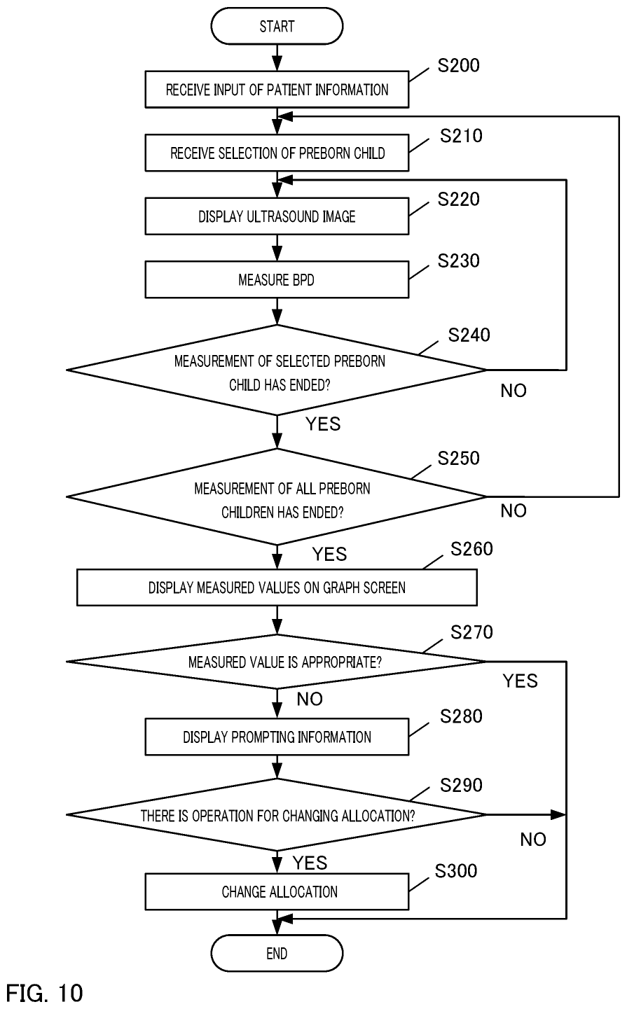

Next, processing of changing the allocation of measured values 430 in Embodiment 2 is described with reference to FIG. 10. Note that. Step S200 to Step S260 in FIG. 10 are the same as Step S100 to Step S160 in FIG. 4, and hence the description thereof is omitted.

In Step S270, preborn child determining section 250 determines whether measured value 430 is appropriate (belongs to the preborn child) on the basis of the first to third conditions. When none of the first to third conditions is satisfied, that is, when it is determined that measured value 430 is appropriate (Step S270: YES), the processing ends. When at least one of the first to third conditions is satisfied, that is, when it is not determined that measured value 430 is appropriate (Step S270: NO), the processing transitions to Step S280.

In Step S280, prompting information display control section 280 causes prompting information to be displayed.

In Step S290, it is determined whether there is an operation for changing the allocation of measured values 430. When there is an operation for changing the allocation (Step S290: YES), the processing transitions to Step S300. Meanwhile, when there is no operation for changing the allocation (Step S290: NO), the processing ends.

In Step S290, allocation input processing section 240 changes the allocation of measured values 430 of the preborn children. In Step S290, when the allocation is changed, as with Embodiment 1, multiple pregnancy development measurement function unit 200 causes item measurement result saving section 300 to record the changed present measured values 430 as present measurement result 310, and causes the allocation change information indicating that the allocation has been changed to be recorded in association with present measured values 430.

Note that, in Embodiment 2 above, determination result revision section 260 that revises the determination result by preborn child determining section 250 may be included. As a result, consistency between the determination result and the allocation change information can be obtained. Preborn child determining section 250 may include a learning function that corrects the boundary for right and wrong in the determination on the basis of the determination result and the allocation change information. As a result, the determination accuracy can be increased.

### Embodiment 3

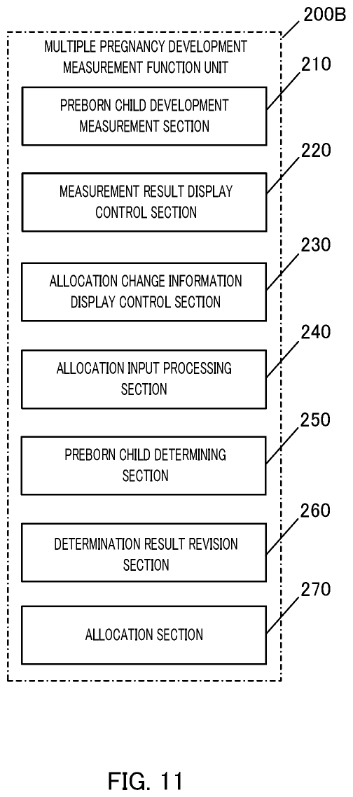

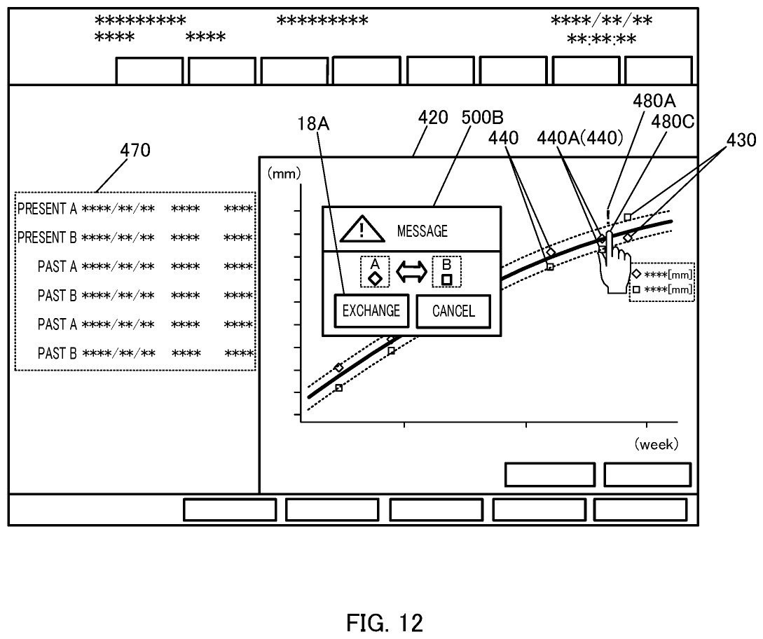

Next, Embodiment 3 of the present invention is described with reference to FIG. 11 and FIG. 12. FIG. 11 is a block diagram illustrating the configuration of multiple pregnancy development measurement function unit 200B. In the description of Embodiment 3, the configurations different from those of Embodiment 1 are mainly described. The same configurations as those of Embodiment 1 are denoted by the same numerals and the description thereof is omitted.

As illustrated in FIG. 11, multiple pregnancy development measurement function unit 200B includes allocation section 270. Allocation section 270 allocates present measured value 430 to one of the plurality of preborn children on the basis of a predetermined condition. The predetermined condition is herein the degree of relevance between present measured value 430 and past measured value 440 of each of the plurality of preborn children.

FIG. 12 illustrates present measured values 430 and previous measured values 440A. When past measured value 440 is a measured value the allocation of which is changed to another preborn child after being allocated to one preborn child on the basis of the predetermined condition, allocation change information display control section 230 causes exclamation mark 480A serving as the allocation change information to be displayed on graph screen 420 indicating that the allocation of the past measured value (previous measured value 440A in FIG. 12) the allocation of which has been changed has been changed.

Note that the user may determine that previous measured values 440A before the change belong to the respective preborn children.

When the user determines that previous measured values 440A before the change belong to the respective preborn children, the user touches graph screen 420. Allocation change information display control section 230 causes index-finger-shaped mouse pointer 480C for changing the allocation of measured values 440A in response to a touch operation on graph screen 420 to be displayed on graph screen 420 (see FIG. 12). Allocation input processing section 240 changes the allocation of measured values 440A in response to the operation of mouse pointer 480C.

According to Embodiment 3, mouse pointer 480C is displayed, and the allocation of measured values 440A can be changed in an easier manner in response to the operation of mouse pointer 480C. Therefore, the time spent in the change of the allocation of measured values 440A is further reduced, and the examination time can be reduced even more.

Alternatively, when the user determines that previous measured values 440A before the change belong to the respective preborn children, the user touches measured values 440A. Allocation change information display control section 230 causes operation key 18A to be displayed on graph screen 420. Allocation input processing section 240 changes the allocation of measured values 440A in response to the operation of operation section 18.

According to this embodiment, the allocation of measured values 440A can be changed in an easier manner by directly selecting measured values 440A that are desired to be changed. Therefore, the time spent in the change of the allocation of measured values 440A is further reduced, and the examination time can be reduced even more.

Meanwhile, preborn child determining section 250 may determine that previous measured values 440A before the change belong to the respective preborn children.

When preborn child determining section 250 determines that previous measured values 440A before the change belong to the respective preborn children, allocation change information display control section 230 causes message 500B (for example, information indicating that the possibility of the allocation of the measured values being erroneously changed is high) and operation key 18A to be displayed on graph screen 420 (see FIG. 12).

According to Embodiment 3 described above, message 500B is displayed, and hence the user can be prompted to visually recognize that the possibility of the allocation of measured values 440A being erroneously changed is high.

In Embodiment 3, the allocation changing operation by operation key 18A can be performed on ultrasound image 490, and hence the change of the allocation can be easily performed.

Note that, in Embodiment 3, the operation for changing the allocation may be performed during the measurement performed by preborn child development measurement section 210 in order to reduce the examination time.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasound diagnostic apparatus, comprising:
a measurer that measures a predetermined item for each of a plurality of preborn children in a body of a same mother;
a display; and
a hardware processor that:
causes the display to display measurement results respectively allocated to the plurality of preborn children,
causes an operation key associated with an operation for changing the allocation of the measurement results to be displayed on a same screen as a screen on which the measurement results are displayed; and
changes the allocation of the measurement results respectively allocated to the plurality of preborn children among the plurality of preborn children in response to an operation of the operation key.

2. The ultrasound diagnostic apparatus according to claim 1, wherein the hardware processor causes allocation change information to be displayed in a case where the allocation of the measurement results of the plurality of preborn children is changed, the allocation change information indicating that the allocation of the measurement results of the plurality of preborn children is changed.

3. The ultrasound diagnostic apparatus according to claim 1, wherein the hardware processor causes the measurement results of the plurality of preborn children to be displayed on a graph screen.

4. The ultrasound diagnostic apparatus according to claim 3, wherein the operation key is displayed on a same screen as the graph screen.

5. The ultrasound diagnostic apparatus according to claim 2, wherein the hardware processor causes the measurement results of the plurality of preborn children to be displayed on a measured value display screen.

6. The ultrasound diagnostic apparatus according to claim 1, wherein the hardware processor causes the measurement results of the plurality of preborn children presently measured by the measurer and the measurement results of the plurality of preborn children measured at or before a previous time by the measurer to be displayed.

7. The ultrasound diagnostic apparatus according to claim 1, wherein the hardware processor causes likelihood information to be displayed, the likelihood information indicating a likelihood of the measurement results belonging to the preborn children.

8. The ultrasound diagnostic apparatus according to claim 1, further comprising a likelihood information recorder that records likelihood information indicating a likelihood of the measurement results belonging to the preborn children.

9. The ultrasound diagnostic apparatus according to claim 2, wherein the hardware processor causes the allocation change information to be displayed on a graph screen.

10. The ultrasound diagnostic apparatus according to claim 5, wherein the hardware processor causes the measured value display screen to display the allocation change information.

11. The ultrasound diagnostic apparatus according to claim 1, wherein the hardware processor changes the measurement results of the preborn children whose allocation is changed to the measurement results of the preborn children before the change again.

12. The ultrasound diagnostic apparatus according to claim 11, wherein the hardware processor changes the allocation of the measurement results of the preborn children on an ultrasound image of the preborn children.

13. The ultrasound diagnostic apparatus according to claim 11, wherein the hardware processor changes the allocation of the measurement results of the preborn children during the measurement of the preborn children performed by the measurer.

14. An ultrasound diagnostic apparatus, comprising:
a measurer that measures a predetermined item for each of a plurality of preborn children in a body of a same mother,
a display; and
a hardware processor that:
causes the display to display measurement results respectively allocated to the plurality of preborn children, the hardware processor determining whether the measurement results of the plurality of preborn children belong to the respective preborn children;
causes the display to display prompting information that prompts a change of the allocation of the measurement results of the plurality of preborn children in accordance with a determination result; and
changes the allocation of the plurality of measurement results among the plurality of preborn children.

15. The ultrasound diagnostic apparatus according to claim 14, further comprising a determination result recorder that records the determination result obtained by determining whether the measure results of the plurality of preborn children belong to the respective preborn children.

16. The ultrasound diagnostic apparatus according to claim 14, wherein the hardware processor revises the determination result.

17. An ultrasound diagnostic apparatus, comprising:
a display; and
a hardware processor that:
automatically allocates measurement results to any one of a plurality of preborn children in a body of a same mother on basis of a predetermined condition in measurement of a predetermined item performed for each of the plurality of preborn children;
causes the display to display the measurement results respectively allocated to the plurality of preborn children;
changes the allocation of the measurement results respectively allocated to the plurality of preborn children among the plurality of preborn children; and
causes allocation change information indicating that the measurement results are not allocated on basis of the predetermined condition to be displayed, once the allocation of the measurement results is changed.

18. The ultrasound diagnostic apparatus according to claim 17, wherein the predetermined condition comprises a degree of relevance between the measurement results of the plurality of preborn children that are presently measured and the measurement results of the plurality of preborn children measured at or before a previous time.

19. The ultrasound diagnostic apparatus according to claim 1, further comprising a memory that stores therein each of the plurality of preborn children and the measurement results in association with each other, wherein the memory stores therein information indicating that the allocation is changed in association with the measurement results in a case where the allocation of the measurement results respectively allocated to the plurality of preborn children is changed among the plurality of preborn children.

20. A non-transitory computer-readable recording medium storing therein a control program for causing a computer of an ultrasound diagnostic apparatus comprising a measurer that measures a predetermined item for each of a plurality of preborn children in a body of a same mother and a display to execute:
causing the display to display measurement results respectively allocated to the plurality of preborn children; and
causing an operation key associated with an operation for changing the allocation of the measurement results to be displayed on a same screen as a screen on which the measurement results are displayed, and changing the allocation of the measurement results respectively allocated to the plurality of preborn children among the plurality of preborn children in response to an operation of the operation key.

21. A non-transitory computer-readable recording medium storing therein a control program for causing a computer of an ultrasound diagnostic apparatus comprising a measurer that measures a predetermined item for each of a plurality of preborn children in a body of a same mother and a display to execute:
causing the display to display measurement results respectively allocated to the plurality of preborn children; and
determining whether the measurement results of the plurality of preborn children belong to the respective preborn children;
causing the display to display prompting information that prompts a change of the allocation of the measurement results of the plurality of preborn children in accordance with a determination result; and
changing the allocation of the plurality of measurement results among the plurality of preborn children.

22. A non-transitory computer-readable recording medium storing therein a control program for causing a computer of an ultrasound diagnostic apparatus comprising a display to execute:
automatically allocating measurement results to any one of a plurality of preborn children in a body of a same mother on basis of a predetermined condition in measurement of a predetermined item performed for each of the plurality of preborn children;
causing the display to display the measurement results respectively allocated to the plurality of preborn children;
changing the allocation of the measurement results respectively allocated to the plurality of preborn children among the plurality of preborn children; and
causing allocation change information indicating that the measurement results are not allocated on basis of the predetermined condition to be displayed, in a case where the allocation of the measurement results is changed.
